(19) United States
(12) Patent Application Publication
(10) Pub. No.: US 2005/0228457 A1
(43) Pub. Date: Oct. 13, 2005

(54) Finishing wire compatible with multiple components

(76) Inventors: Mo Jafari, Murrieta, CA (US); John Greenland, San Diego, CA (US); Paul Haldeman, Murrieta, CA (US); Charles Robert Peterson, Murrieta, CA (US)

Correspondence Address: SCHWEGMAN, LUNDBERG, WOESSNER & KLUTH, P.A., P.O. BOX 2938, MINNEAPOLIS, MN, 55402-0938, US

(21) Appl. No.: 10/819,704

(22) Filed: Apr. 7, 2004

(51) Int. Cl. A61N 1/372

(52) U.S. Cl. 607/37

(57) ABSTRACT

A finishing wire assembly employs features that allow for the finishing wire assembly to be utilized with several different types of leads having terminal pins with varying structure, such as different outer or inner diameters for the terminal pins.

### TECHNICAL FIELD

Lead assemblies for conducting electrical signals to and from a location such as a heart, and more particularly, a wire and lead assembly allowing for placement, manipulation, or removal of placement devices.

### TECHNICAL BACKGROUND

Pacemaker leads represent the electrical link between the pulse generator and the heart tissue, which is to be excited and/or sensed. These pacemaker leads include single or multiconductors that are connected to an electrode in an electrode assembly at an intermediate portion or distal end of a pacing lead. A connector is included at the proximal end to form the electrical connection with the pacemaker.

To implant the lead within the patient, the lead is often fed intravenously toward the heart. The lead may be implanted within or travel through complex or tortuous vasculature using, for example, a guide catheter. After the lead has been placed in the desired position, the guide catheter must be extracted. However, extracting the guide catheter can cause the lead to be dislodged from the implanted position. A finishing wire can be used to assist in maintaining a lead in its implanted position during removal of the guide catheter. However, with leads having varying lengths, and varying terminal pins, an individual finishing wire is required for each different lead type.

Accordingly, there is a need for a finishing wire assembly that can accommodate different sized leads having different features, such as different terminal pins.

### SUMMARY

A finishing wire assembly employs features that allow for the finishing wire assembly to be utilized with several different types of leads having terminal pins with varying structure, such as different outer or inner diameters for the terminal pins. In one option, the finishing wire assembly includes a finishing wire and a portion of the finishing wire that deviates from the longitudinal axis, where the deviating portion is coupleable with an interior surface of a lead terminal pin.

Several options for the finishing wire assembly are as follows. For instance, in one option, the finishing wire includes a stop disposal on the finishing wire, which prevents over insertion of the finishing wire assembly. In another option, a coupling, such as a hypo tube, is coupled with the finishing wire so that the assembly can be coupled with the lead terminal pin. The finishing wire, in another option, includes one or more deformations within the finishing wire, which have outer dimensions, in one option, at least slightly greater than an inner diameter of the terminal pin.

In another embodiment, the finishing wire assembly includes a finishing wire and a coupling secured along a portion of the finishing wire, where the coupling can be coupled with a terminal pin of the lead assembly. The coupling includes a first coupling portion that has a first inner dimension and a second coupling portion with a second inner dimension, where the first dimension is greater than the second inner dimension.

Several options for the finishing wire assembly are as follows. For instance, the first coupling is disposed at a different longitudinal location than the second coupling portion. In another option, the second coupling portion or the first coupling portion include one or more projections extending from an inner surface of the coupling. In another option, the second coupling portion or the first coupling portion include one or more dimples extending from an outer surface of the coupling in to the inner surface of the coupling. In another option, the coupling is formed from a polymer material, that optionally includes an internal stepped portion, that allows for the coupling to mate with pins having a variety of outer diameters or structures.

A method is further provided that includes advancing a guide catheter in a lead, inserting a finishing wire assembly into the lead, where the finishing wire assembly includes a coupling that is coupleable with two or more different terminal pins that have two or more different structures, for example, a different inner structure, and/or a different outer structure of the terminal pin. The method further includes coupling the coupling of the finishing wire assembly with a portion of the terminal pin of the lead, for example, within the inner portion of the terminal lead and/or with the outer portion of the terminal pin of the lead. The method further includes removing the guide catheter while the finishing wire assembly is disposed within the lead.

Several options for the method are as follows. For instance, in one option, coupling the finishing wire assembly with the terminal pin includes resiliently coupling the coupling with the terminal pin. In another option, the method further includes preventing over insertion of the finishing wire assembly within the lead. In yet another option, coupling of the coupling with the terminal pin includes engaging the terminal pin with projections within the finishing wire assembly.

These and other embodiments, aspects, advantages, and features will be set forth in part in the description which follows, and in part will become apparent to those skilled in the art by reference to the following description and referenced drawings or by practice thereof. The aspects, advantages, and features are realized and attained by means of the instrumentalities, procedures, and combinations particularly pointed out in the appended claims and their equivalents.

### BRIEF DESCRIPTION OF THE DRAWINGS

FIG. 1 is a diagram illustrating a lead assembly, a finishing wire assembly, and a guide catheter implanted within a heart.

FIG. 2 illustrates a portion of a finishing wire assembly constructed in accordance with one embodiment.

FIG. 3 illustrates a portion of a lead, and a finishing wire assembly constructed in accordance with one embodiment.

FIG. 4 illustrates a portion of a lead, and a finishing wire assembly constructed in accordance with one embodiment.

FIG. 5 illustrates a perspective view of a finishing wire assembly constructed in accordance with one embodiment.

FIG. 6 illustrates a perspective view of a finishing wire assembly constructed in accordance with one embodiment.

FIG. 7 illustrates a perspective view of a finishing wire assembly constructed in accordance with one embodiment.

FIG. 8 illustrates a perspective view of a finishing wire assembly constructed in accordance with one embodiment.

### DESCRIPTION OF THE EMBODIMENTS

In the following detailed description, reference is made to the accompanying drawings, which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the spirit and scope of the present invention. Therefore, the following detailed description is not to be taken in a limiting sense, and the scope is defined by the appended claims.

A finishing wire assembly is provided herein that employs features that allow for the finishing wire assembly to be utilized with several different types of leads. For example, the finishing wire assembly can be used with leads having a variety of different features at the proximal end that couple with the finishing wire assembly, or other features that vary from lead to lead. The finishing wire assembly is used to stabilize a lead implanted within a patient, for example, within a heart, while a guide catheter is removed over the lead.

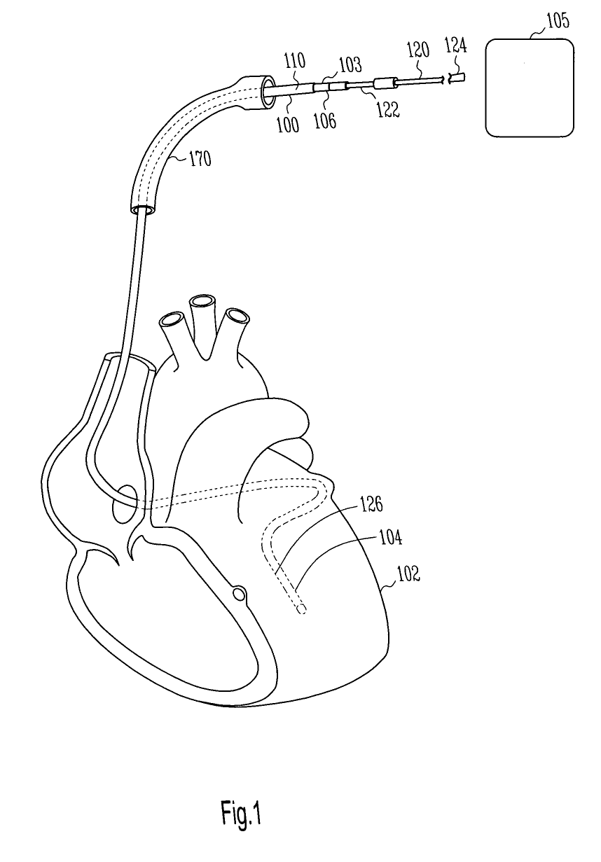

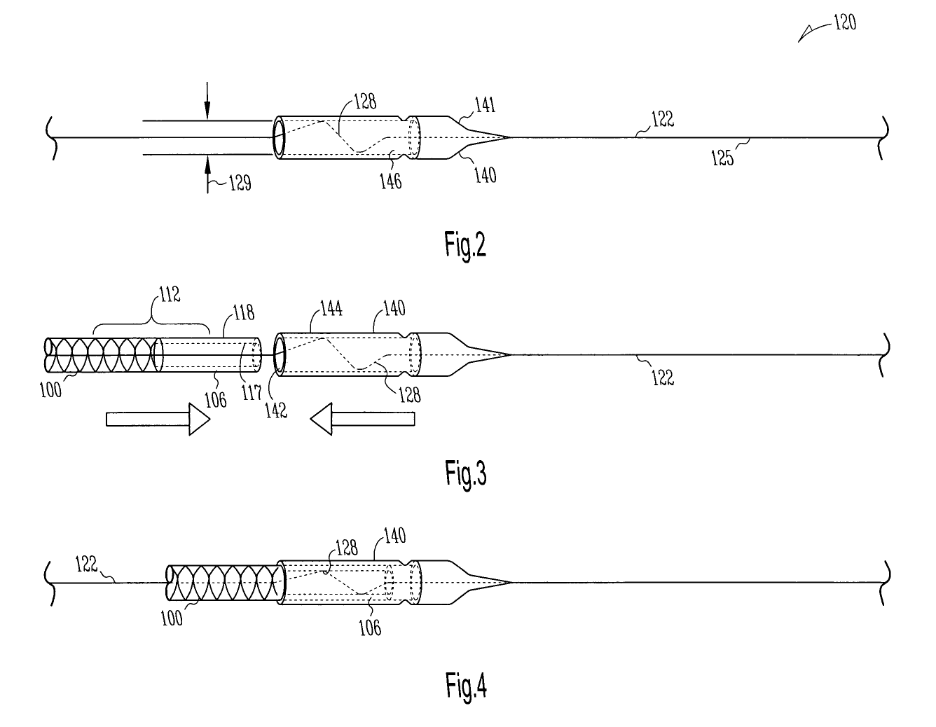

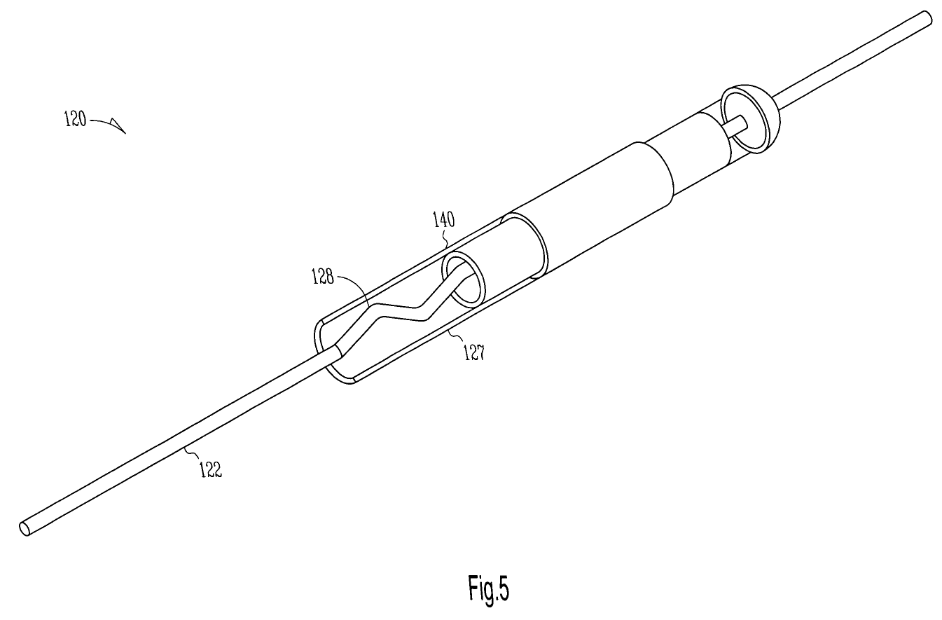

FIG. 1 illustrates a lead assembly constructed in accordance with one embodiment. A lead 100 includes a lead body 110 that is one option implanted in, on, or around a portion of a heart 102. The lead 100 extends from a proximal end 103 to a distal end 104 and provides a conduit of electrical signals in between the heart, and/or tissue to be excited and an energy source 105 such as a pulse generator. A terminal 106 provides the electrical connection and physical connection between the lead 100 and the energy source 105. The terminal 106, illustrated in greater detail in FIG. 3 is disposed at the proximal end 112 of the lead 100, and is defined in part by an inner surface 117 and an outer surface 118. The terminal pins for leads vary, including, but not limited to, IS-1 or LV-1 connectors. Terminal pins further vary from lead to lead, for example, in the outer surfaces 118 and or the inner surfaces 117 of the terminal pins 106.

To implant the lead 100 within a heart 102, a guide catheter 170 is used. It should be noted other types of devices can be used as well, and are considered within the scope of this application. After the lead 100 is implanted with the guide catheter 170, the guide catheter 170 is to be removed from the patient. However, before removal, the guide wire is removed from the lead 100, and a finishing wire assembly 120 is disposed within the lead 100 to maintain the lead in position, as further discussed below, during removal of the guide catheter over the finishing wire assembly 120.

The implantable assembly further includes a finishing wire assembly 120, illustrated in greater detail in FIGS. 2-4. The finishing wire assembly 120 includes a finishing wire 122 and optionally a coupling 140. The finishing wire 122 extends from a proximal end 124 to a distal end 126. When the finishing wire assembly 120 is disposed within the lead 100, the distal end 126 of the finishing wire 122 is disposed proximal to the distal end 104 of the lead 100. However, the finishing wire 122, in one option, is not disposed through the distal end 104 of the lead 110. The finishing wire assembly 120 further includes a coupling 140 for connecting the assembly with the lead.

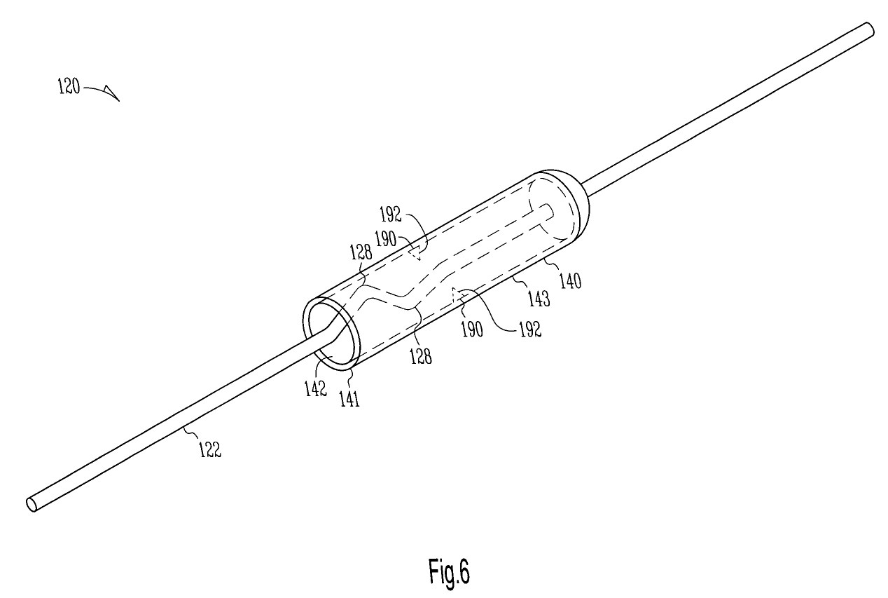

The coupling 140, in one option, includes a cup shaped portion 141 that is attached to the wire 122. Suitable methods for attaching the housing 141 to the wire 122 include, but are not limited to, soldering, glue processes, crimping, or other mechanical attachment methods. In one option, the coupling 140 is formed from a hypo tube 127 (FIG. 5), and is sized to fit within the guide catheter, for example, as the guide catheter is pulled over the finishing wire assembly 120. The coupling 140 is defined in part by an inner surface 142 and an outer surface 144. The inner surface 142 mates with an outer surface 118 of the terminal 106, and optionally provides a stop 146 for the distal end of the terminal 106, thereby preventing over-insertion of the finishing wire 122 within the lead 100 (FIG. 1).

The finishing wire 122 is defined in part by a finishing wire longitudinal axis 125, where the axis 125 is defined while the finishing wire 122 is in a relatively straightened position. The finishing wire 122 includes, in one option, portions which deviate from the axis 125 of the finishing wire 122, for example, which allow for the portions to have a wider dimension than the finishing wire 122 itself. The portions form a portion of the coupling 140, or form its own coupling 140 independent of the portion 141. In one option, the finishing wire 122 includes one or more deformations 128, where the one or more deformations 128 deviate from the finishing wire longitudinal axis 125. In one option, the one or more deformations 128 extend within a single plane, for example, as illustrated in FIG. 2. In another option, the one or more deformations 128 extend within two or more different planes. The deformations 128 can be formed within the finishing wire 122, for example, by a coining operation. In yet another option, other features can be added to the finishing wire to add width to the finishing wire, for example, a piece of material can be secured along the finishing wire 122 to provide width.

The one or more deformations 128 optionally extend along a length of the finishing wire 122 within the coupling 140, or can extend beyond a length of the coupling 140. In yet another option, the one or more deformations 128 have a width 129 that is at least slightly larger than the inner surface 117, for example the inner diameter, of the terminal 106. The deformations of the finishing wire 122 allow for the finishing wire 122 to deform and provide resilience to the finishing wire 122. This also allows for the finishing wire 122 to be coupled with an inner surface of the terminal 106, for example, resiliently coupled, as illustrated in FIG. 4. This further allows for the same finishing wire assembly 120 to be coupled with leads having different terminal pins, where the terminal pins have similar inner surfaces or structures.

As the finishing wire assembly 120 is disposed within the lead 100, the finishing wire 122 begins to straighten to allow the finishing wire 122 to be disposed within the terminal 106. The one or more deformations 128 frictionally engage the inner surface 117 of the terminal 106, and assist in coupling the finishing wire assembly 120 to the lead 100 (FIG. 1), for example, during the removal of the guide catheter. Having the finishing wire 122 engage the inner surface 117 of the terminal 106 allows for the finishing wire assembly 120 to be used with multiple different terminals that have different outer configurations, such as IS-1 and LV-1 terminal pins.

In another option, the finishing wire assembly 120 further includes features that allow the finishing wire assembly 120 to temporarily lock the lead 100 at the distal end 102 (FIG. 1) of the lead 100 such that the guide catheter 170 (FIG. 1) to further allow the lead 100 to be removed without the lead 100 being dislodged. Suitable examples of ways to temporarily lock or restrict the lead 100 at the distal end 102 (FIG. 1) of the finishing wire assembly 120, include but are not limited to, that described in U.S. Pat. No. 6,356,791 issued to Westlund, and U.S. Pat. No. 6,671,560, each issued to Westlund, each assigned to the Cardiac Pacemakers, Inc, and each incorporated by reference herein.

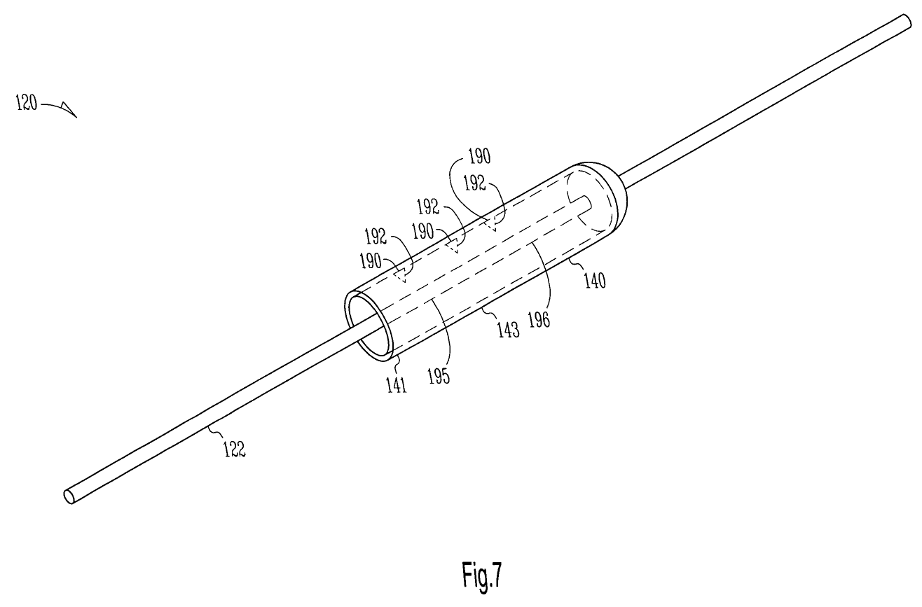

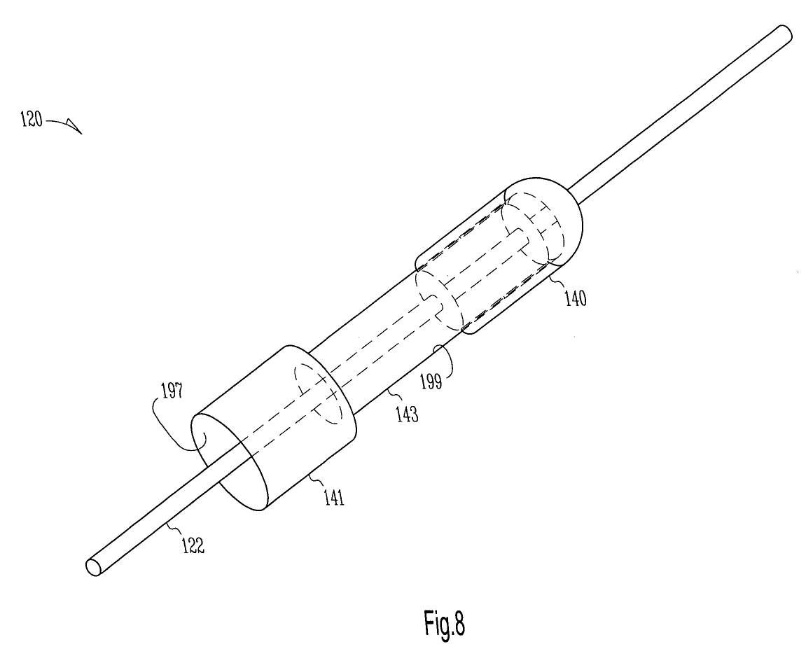

FIGS. 6 and 7 illustrate another example of a finishing wire assembly 120 that can be used with multiple different leads. The finishing wire assembly 120 optionally includes the features discussed above, including the deformations 128 and the coupling 140, where the deformations 128 couple or otherwise engage an inner surface of a terminal pin. In another option, the coupling 140 further includes features 190 formed on the inner surface 142 of the coupling 140 that mate or otherwise engage an outer surface of a terminal pin. The features 190, in one option, are provided in addition to the deformations 128. Alternatively, the features 190 can be provided independently (see FIG. 7).

The features 190 form projections 192 that extend from the inner surface 142 of the coupling 140, and allow for the inner surface of the coupling 140 to have two or more dimensions, allowing for the coupling 140 to be used with two or more different terminal pins having two or more different outer diameters. The projections 192 are formed within the inner surface 142 of the coupling 140, for example, by compressing a portion of the outer surface of the coupling 140 to recess within the coupling 140 and project from the inner surface. In one option, the outer surface of the coupling 140 is compressed while a tube or mandrel is disposed within the coupling 140. For examples, dimples are formed in the outer surface of the coupling 140 which forms the projections on the inner surface of the coupling. The projections can be formed at varying heights to accommodate terminal pins having different outer diameters, or different outer structures, as further described below. Alternatively, the projections 192 can be formed by molding the coupling 140 from material with the projections in place. In yet another option, inserts can be coupled with a portion of the coupling 140 to extend from the inner surface 142.

The projections 192 and/or the dimples are positioned along an intermediate portion 143 of the coupling 140 such that a distal end 141 of the coupling 140 is suitable for use with a terminal pin having a first outer diameter, and the intermediate portion, with the projections 192, are suitable for use with a terminal pin having a second outer diameter, and the first outer diameter is greater than the second outer diameter. For example, a first terminal pin of a first lead with a first outer diameter would be coupled with the coupling 140 along the distal end 141 of the coupling 140 with the inner surface 142 and/or the deformations. A second terminal pin of a second lead having a second outer diameter that is smaller than the first lead is coupled at the intermediate portion 143 of the coupling 140 with the projections 192 that engage the outer surface of the terminal pin.

The height of the projections 192, and/or the depth of the dimples at the distal end, in one option, is different than the height of the projections 192 and/or the depth of the dimples at the intermediate portion 143 of the terminal pin, thereby accommodating at least two different terminal pins. In one option, the height of the projections 192 and/or the depth of the dimples at the intermediate portion 143 is larger than the height of the projections 192 and/or the dimples at the distal end of the terminal pin.

FIG. 7 illustrates a further option of the finishing wire assembly 120 which allows for the assembly to be used with leads having different terminal pins, for example, terminal pins having two or more different outer diameters. The finishing wire assembly 120, in one option, includes two or more features having two or more inner dimensions within the coupling 140. For example, the coupling 140 includes a first set of projections 196 disposed along an intermediate portion 143 of the coupling 140, and at least a second set of projections 195 disposed proximate to the distal portion 141 of the coupling 140. Although the term “set” is used, it should be noted that one projection can be provided, and further the projections can have a variety of shapes to achieve the resulting inner diameters accommodating the terminal pins with the varying outer diameters.

In one option, the first set of projections 196 form an inner dimension that mates with a terminal pin having an outer diameter that is smaller than a terminal pin that mates with the second set of projections 195. For example, in one option, the first set of projections 196 extend within the inner surface of the coupling 140 to a depth that engages an outer surface of an IS-1 terminal pin. The second set of projections 195 extend within the inner surface of the coupling 140 to a depth that engages an outer surface of an LV-1 terminal pin. Thus, the coupling 140 would be compatible for use with leads that have either LV-1 terminal pins or IS-1 terminal pins.

FIG. 8 illustrates another embodiment of the finishing wire assembly 120. It should be noted that the features of FIG. 8 can be combined with various features discussed above, and/or illustrated in the figures. The coupling 140 includes a first inner diameter 197 formed along the distal end 141 of the coupling 140, and a second inner diameter 199 formed along the intermediate portion 143 of the coupling 140, where the first inner diameter 197 is greater than the second inner diameter 199. In one option, at least one of the first inner diameter 197 or the second inner diameter 199 forms an annular step within the coupling 140.

The coupling is formed in one option from a polymer material, that optionally includes an internal stepped portion, that allows for the coupling to mate with pins having a variety of outer diameters or structures. The polymer coupling can be thermally formed, molded, or extruded, for example, in separate pieces. In another option, the coupling is formed of a metal, such as berylium, copper, stainless steel.

In another option, any of the above-discussed and/or illustrated finishing wire assemblies can include features that allow for the insertion length to be modified. For example, the coupling 140 can be movably coupled with the finishing wire 122, and secured along the finishing wire 122 in multiple locations, as further discussed in co-pending application Ser. No. ______ entitled “FINISHING WIRE ASSEMBLY HAVING VARIABLE INSERTION LENGTH AND METHOD THEREFOR”, attorney docket no. 279.730US1, filed on ______. This application is assigned to Cardiac Pacemakers, Inc., and is incorporated by reference herein in its entirety.

A method of using the implantable assembly is described herein. A guide catheter and optionally and guidewire is used to implant a lead within a patient, for example, within a heart. After the guidewire is used to insert the lead, the guidewire is withdrawn and a finishing wire is inserted into the lead. In one option, a distal end of the finishing wire is coupled or otherwise temporarily locked with a distal portion of the lead. Suitable examples of ways to temporarily lock or restrict the lead 100 at the distal end 102 of the finishing wire 122, include but are not limited to, that described in U.S. Pat. No. 6,356,791 issued to Westlund, and U.S. Pat. No. 6,671,560, each issued to Westlund, each assigned to the Cardiac Pacemakers, Inc, and each incorporated by reference herein.

A proximal portion of the finishing wire assembly is coupled with the terminal of the lead, and is coupleable with two or more different terminal pins having a variety of structures. For example, the finishing wire assembly is coupleable with a lead having a first type of terminal pin, such as an LV-1 terminal pin. The same finishing wire assembly is coupleable with a lead having a second type of terminal pin, such as an IS-1 terminal pin.

In one option, the finishing wire is coupled with an inner portion of the terminal of the lead, for instance, as discussed above. In one example, the finishing wire resiliently engages an inner surface of the terminal pin. Alternatively, or in addition, the finishing wire assembly is coupled with an outer surface of the terminal pin, for example, through use of a coupling of the finishing wire assembly. For instance, the coupling includes one or more portions on the inner surface of the coupling member that allows for selective connection or coupling with the outer surface of the terminal pin, as further discussed above.

The guide catheter is removed from over the lead and over the finishing wire. The finishing wire retains the lead in place during extraction of the surrounding guide catheter. Following removal of the guide catheter, the finishing wire is removed from the lead, for example, by simply pulling out the finishing wire while holding the proximal end of the lead to prevent it from being dislodged from the position in which the lead had been placed.

Advantageously, the finishing wire assembly eliminates the need of having multiple finishing wire assemblies provided when multiple leads having different dimensions are provided. For example, a single finishing wire assembly can be provided in a kit with multiple leads, where the lead has not been pre-selected. The single finishing wire assembly can be used with a lead having an IS-1 terminal pin, and the same finishing wire assembly can be used with a lead having an LV-1 terminal pin. This will reduce confusion between the multiple parts, as well as inventory needed for each procedure. Furthermore, the assembly allows for the insertion force of the assembly into the lead to be comparable to the extraction force, when the assembly is removed from the lead. This prevents further disruption to the lead once it is implanted.

It is to be understood that the above description is intended to be illustrative, and not restrictive. Although the use of the implantable device has been described for use as a lead in, for example, a cardiac stimulation system, the implantable device could as well be applied to other types of body stimulating systems. Many other embodiments will be apparent to those of skill in the art upon reviewing the above description. The scope should, therefore, be determined with reference to the appended claims, along with the full scope of equivalents to which such claims are entitled.

1. A finishing wire assembly comprising:
a finishing wire extending from a finishing wire proximal end to a finishing wire distal end, the finishing wire including a proximal portion near the finishing wire proximal end;
the proximal portion including a portion deviating from a central longitudinal axis defined by a longitudinal axis of a straightened finishing wire;
the portion deviating from the central longitudinal axis coupleable with an interior surface of a lead terminal pin.

2. The finishing wire assembly as recited in claim 1, further comprising a stop disposed along the finishing wire, the stop preventing over-insertion of the finishing wire assembly.

3. The finishing wire assembly as recited in claim 1, further comprising a coupling coupleable with the lead terminal pin.

4. The finishing wire assembly as recited in claim 1, wherein the coupling includes a hypotube secured along the finishing wire.

5. The finishing wire assembly as recited in claim 1, wherein the coupling is coupleable with at least two different terminal pins having two different outer dimensions.

6. The finishing wire assembly as recited in claim 1, wherein the portion deviating from a central longitudinal axis includes one or more deformations in the finishing wire.

7. The finishing wire assembly as recited in claim 6, wherein the one or more deformations have an outer dimensions at least slightly greater than an inner diameter of the terminal pin.

8. A finishing wire assembly comprising:
a finishing wire extending from a finishing wire proximal end to a finishing wire distal end, the finishing wire including a proximal portion near the finishing wire proximal end;
a coupling secured along a portion of the finishing wire, the coupling coupleable with a terminal pin of a lead assembly; and
the coupling including a first coupling portion having a first inner dimension and a second coupling portion having a second inner dimension, and the first inner dimension is greater than the second inner dimension.

9. The finishing wire assembly as recited in claim 8, wherein the first coupling portion is disposed along a first longitudinal location along the coupling and the second portion is disposed along a second longitudinal location different than the first longitudinal location.

10. The finishing wire assembly as recited in claim 8, wherein at least the second coupling portion includes one or more projections extending from an inner surface of the coupling.

11. The finishing wire assembly as recited in claim 8, wherein at least the first coupling portion includes one or more projections extending from an inner surface of the coupling.

12. The finishing wire assembly as recited in claim 8, wherein at least one of the first coupling portion or the second coupling portion includes an annular step.

13. The finishing wire assembly as recited in claim 8, wherein the finishing wire is sized or deformed to be engageable with an inner surface of the terminal pin.

14. The finishing wire assembly as recited in claim 13, wherein the finishing wire includes one or more deformations therein.

15. The finishing wire assembly as recited in claim 8, wherein the coupling is formed from polymer material and includes a stepped inner surface.

16. The finishing wire assembly as recited in claim 8, wherein at least the second coupling portion includes one or more projections extending from an inner surface of the coupling.

17. A method comprising:
advancing a guide catheter and a lead;
inserting a finishing wire assembly into the lead, the finishing wire assembly including a coupling coupleable with two or more different terminal pins having two or more different structures;
coupling the coupling of the finishing wire assembly with a terminal pin of the lead; and
removing the guide catheter while the finishing wire assembly is disposed within the lead.

18. The method as recited in claim 17, further comprising deforming a portion of the finishing wire assembly.

19. The method as recited in claim 17, wherein coupling the coupling of the finishing wire assembly with the terminal pin includes engaging a finishing wire of the finishing wire assembly with an inner surface of the terminal pins.

20. The method as recited in claim 17, wherein coupling the coupling of the finishing wire assembly with the terminal pin includes coupling the terminal pin with at least one of a first portion or a second portion of the coupling, and the first portion has a different inner dimension than the second portion.

21. The method as recited in claim 17, wherein coupling the coupling of the finishing wire assembly with the terminal pin includes resiliently coupling the coupling with the terminal pin.

22. The method as recited in claim 17, wherein coupling the coupling of the finishing wire assembly with the terminal pin includes engaging the terminal pin with projection of the coupling.

23. The method as recited in claim 17, further comprising preventing over-insertion of the finishing wire assembly within the lead.
